(19) United States
(12) Patent Application Publication
(10) Pub. No.: US 2025/0041074 A1
(43) Pub. Date: Feb. 6, 2025

(54) MEDICAL IMPLANT DEVICE

(71) Applicant: Statera Medical Inc., Montreal (CA)

(72) Inventors: Frédérik Plourde, Montreal (CA); Samuel Bourdon, Montreal (CA); Marc Blondin, Montreal (CA); Moreno Morelli, Montreal (CA)

(73) Assignee: Statera Medical Inc., Montreal, QU (CA)

(21) Appl. No.: 18/837,086

(22) PCT Filed: Apr. 21, 2023
(86) PCT No.: PCT/IB2023/054123

§ 371 Date: Aug. 8, 2024

Related U.S. Application Data
(60) Provisional application No. 63/334,957, filed on Apr. 26, 2022.

(51) Int. Cl. A61F 2/46; A61F 2/30

(52) U.S. Cl. CPC A61F 2/46; A61F 2002/3071

(57) ABSTRACT

Systems and methods to evaluate a joint implant device are provided. The joint implant device can include a body having a first end to couple with a first joint component and a second end to couple with a second joint component. The body can include a first gear mechanism to cause a change in axial distance between the first end and the second end of the body. The body can include a second gear mechanism to cause a change in angle between the first end and the second end of the body about an axis.

### CROSS-REFERENCE TO RELATED APPLICATIONS

This application claims the benefit of and priority to U.S. Provisional Patent Application No. 63/334,957, filed Apr. 26, 2022, the entire disclosure of which is incorporated by reference herein.

### BACKGROUND

Various medical devices can be used to support several bones, muscles, ligaments, or tendons in a body.

### SUMMARY

At least one aspect is directed to a joint implant device. The joint implant device can include a body. The body can include a first end to couple with a first joint component and a second end to couple with a second joint component. The body can include a first gear mechanism to cause a change in axial distance between the first end and the second end of the body. The body can include a second gear mechanism to cause a change in angle between the first end and the second end of the body about an axis.

At least one aspect is directed to a joint implant device. The joint implant device can include a prosthesis body having a first end to couple with a first joint component and a second end to couple with a second joint component. The prosthesis body can include a first set of gears coupled with a first axle, a second set of gears coupled with a second axle, a third set of gears coupled with a third axle, or a fourth set of gears coupled with a fourth axle. Synchronous rotation of the first axle and the second axle can cause a change in axial distance between the first end and the second end. Asynchronous rotation of the first axle and the second axle can cause a change in angle between the first end and the second end about a first axis. Rotation of the third axle and rotation of the fourth axle can cause a change in angle between the first end and the second end about a second axis that is perpendicular to the first axis.

At least one aspect is directed to a method. The method can include receiving, by an internal module of a joint implant device, a first signal from an external module to activate the internal module. The method can include detecting, via the internal module of the joint implant device, a physical metric of the joint implant device. The method can include receiving, via the external module, a second signal from the internal module in response to the first signal. The method can include obtaining, via an input component of a data processing system, data based on the second signal corresponding to the physical metric detected by the internal module of the joint implant device from the external module communicably coupled with the internal module of the joint implant device. The method can include determining, via an adjustment component of the data processing system, an indication to adjust at least one of a thickness or an orientation of the joint implant device based on the second signal.

At least one aspect is directed to a joint implant device. The joint implant device can include a prosthesis body having a first end to couple with a first joint component and a second end to couple with a second joint component. The prosthesis body can include a first axle having a first worm gear and a second worm gear each coupled with the first axle. The prosthesis body can include a second axle having a third worm gear and a fourth worm gear each coupled with the second axle. The prosthesis body can include a third axle having a fifth worm gear and a sixth worm gear each coupled with the third axle. The prosthesis body can include a fourth axle coupled with the third axle and having a seventh worm gear and an eighth worm gear each coupled with the fourth axle. The prosthesis body can include the first worm gear rotatably coupled with a first worm wheel, the second worm gear rotatably coupled with a second worm wheel, the third worm gear rotatably coupled with a third worm wheel, the fourth worm gear rotatably coupled with a fourth worm wheel, the fifth worm gear rotatably coupled with a fifth worm wheel, the sixth worm gear rotatably coupled with a sixth worm wheel, the seventh worm gear rotatably coupled with a seventh worm wheel, and the eighth worm gear rotatably coupled with an eighth worm wheel. The prosthesis body can include the first worm wheel coupled with the second end of the prosthesis body and having a first threaded female mating portion, the second worm wheel coupled with the second end of the prosthesis body and having a second threaded female mating portion, the third worm wheel coupled with the second end of the prosthesis body and having a third threaded female mating portion, and the fourth worm wheel coupled with the second end of the prosthesis body and having a fourth threaded female mating portion. The prosthesis body can include the fifth worm wheel coupled with the first end of the prosthesis body and having a first threaded male mating portion, the sixth worm wheel coupled with the first end of the prosthesis body and having a second threaded male mating portion, the seventh worm wheel coupled with the first end of the prosthesis body and having a third threaded male mating portion, and the eighth worm wheel coupled with the first end of the prosthesis body and having a fourth threaded male mating portion. The first worm wheel and the fifth worm wheel can movably couple with one another by the first threaded female mating portion and the first threaded male mating portion. The second worm wheel and the sixth worm wheel can movably couple with one another by the second threaded female mating portion and the second threaded male mating portion. The third worm wheel and the seventh worm wheel can movably couple with one another by the third threaded female mating portion and the third threaded male mating portion. The fourth worm wheel and the eighth worm wheel can movably couple with one another by the fourth threaded female mating portion and the fourth threaded male mating portion.

At least one aspect is directed to a method. The method can include rotating a first end of a body about a first axis by rotating a first axle. The method can include rotating the first end of the body about a second axis by rotating a third axle. The method can include changing a thickness of the body by rotating the first axle and a second axle simultaneously.

At least one aspect is directed to a multi-axis adjustable joint implant device. The device can include a body having a first end to couple with a first joint component and a second end to couple with a second joint component. The body can include at least one set of gears coupled with at least one corresponding axle. The body can include means for causing a change in axial distance between the first end and the second end of the body. The body can include means for causing a change in angle between the first end and the second end of the body about a first axis. The body can include means for causing a change in angle between the first end and the second end of the body about a second axis that is perpendicular to the first axis.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is an example exploded view of a joint implant device, in accordance with an implementation.

FIG. 2 is an example exploded view of a portion of the joint implant device of FIG. 1, in accordance with an implementation.

FIG. 3 is an example perspective view of a portion of the joint implant device of FIG. 1 in a first example position, in accordance with an implementation.

FIG. 4 is an example perspective view of a portion of the joint implant device of FIG. 1 in a second example position, in accordance with an implementation.

FIG. 5 is an example schematic of a joint implant device system, in accordance with an implementation.

FIG. 6 is an example schematic of a portion of the system of FIG. 5, in accordance with an implementation.

FIG. 7 is an example of a graphical user interface, in accordance with an implementation.

FIG. 8 is an example perspective view of a portion of the joint implant device of FIG. 1, in accordance with an implementation.

FIG. 9 is an illustration of an example method of evaluating a joint implant device, in accordance with an implementation.

### DETAILED DESCRIPTION

Following below are more detailed descriptions of various concepts related to, and implementations of, methods, apparatuses, and systems of medical devices. The various concepts introduced above and discussed in greater detail below may be implemented in any of numerous ways.

The present disclosure generally relates to systems and methods for providing and evaluating a joint implant device. The joint implant device can be used in joint replacement operations to support or replace joints. The technical solution is generally directed to a medical device. For example, this technical solution is generally directed to a joint implant device for coupling with a joint of a human body such as a knee joint, shoulder joint, hip joint, ankle joint, or elbow joint. Generally, joints of a human body, such as the knee joint, are complex in nature. During joint replacement or reparation surgery, restoring the center of rotation may include evaluating soft tissue tension and balance about the joint, as soft tissues play an important role in optimizing joint function for joint replacement surgery.

Current joint replacement implants focus on bone anatomy and alignment without addressing soft tissue tension. Conventional implants include a fixed inclination and do not have means for adjusting post-operation to reach a desired height, tilt, inclination, or rotation of the implant. Thus, achieving adequate soft tissue tension is difficult and there is a need for implants that can be adjusted in multiple planes (i.e., height, version, tilt/inclination, and rotation).

This is a particularly important with knee implants, as it is very difficult to reproduce the natural biomechanical properties of the knee joint. Post-operative function following a knee replacement depends on sufficient balancing of both the flexion and extension gaps and coronal (medial/lateral) gaps. Thus, adjustment and effective fine tuning of these gaps intraoperatively may be needed. A well-balanced knee leads to better functional outcomes and potentially survivorship of the implants.

This technical solution provides an adjustable prosthesis and user interface to provide surgeons with objective data regarding an orientation, location, and amplitude of force on the prosthesis. Furthermore, if the orientation or level of force is not adequate, the prosthesis can be adjusted on multiple planes with a simple action such as turning a hand tool. This technical solution also provides operative or post-operatively measuring data and adjusting the prosthesis with a minimally invasive procedure or non-invasive procedure, reducing discomfort and risk for the patient.

Further, this technical solution may have many benefits over existing medical device evaluating systems. For example, by determining objective data measurements using various computing devices, the technical solution may provide more precise physical metrics of a medical device in comparison to manual evaluation techniques. Furthermore, since typically known evaluation and adjustment techniques are typically generated manually, such adjustments are often derived based on subjective data on a case-by-case and practitioner-by practitioner basis. Additionally, allowing adjustment of the implant on multiple planes through which a joint moves or rotates may help restore joint kinematics that is otherwise difficult or impossible to achieve with conventional systems. This technical solution provides for real-time, repeatable, and accurate evaluation outcomes to more efficiently and effectively adjust a medical device to meet patient needs. Various other technical benefits and advantages are described in greater detail herein.

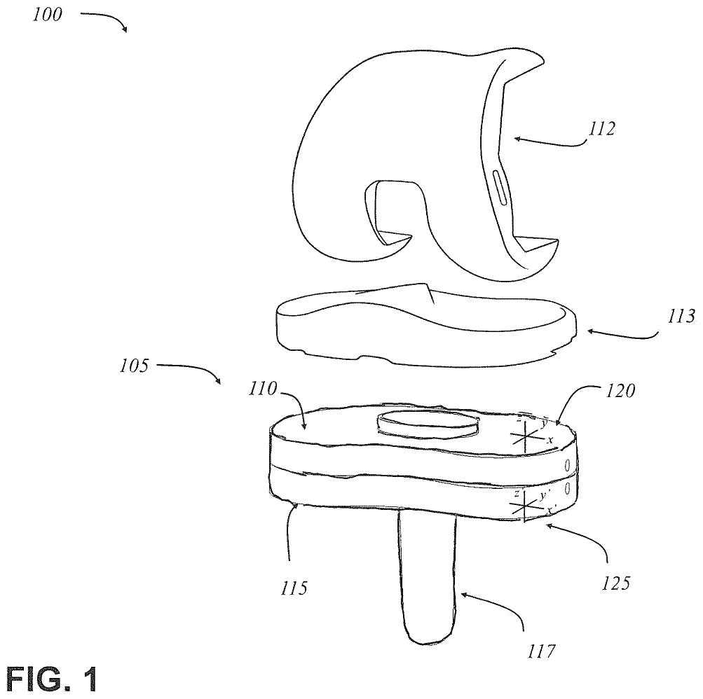

The present disclosure generally relates to a joint implant device that is adjustable about multiple axes. For example, the joint implant device can be adjustable in at least thickness (e.g., a longitudinal length of the body described herein, along the z-axis or z′-axis as shown in FIG. 1 and described herein, a distance between two ends of the body described herein) as well as the relative orientation between two ends of the device along multiple axes, allowing the body to be adjustable and having at least two degrees of freedom.

FIG. 1 illustrates an example perspective exploded view of a joint implant device 100 according to an implementation. For example, the joint implant device 100 can couple with various joints in a body such as a knee joint, a shoulder joint, a hip joint, an ankle joint, a wrist joint, an elbow joint, or another joint. While the joint implant device 100 described in reference to the figures generally relates to a knee joint, the joint implant device 100 can be used with various other joints.

The joint implant device 100 can include at least one body 105, as shown in FIGS. 1-4 and 8, and among others. For example, the body 105 can include a prosthesis body (e.g., an artificial body part). The body 105 can include at least one component. For example, the body 105 can include at least two components (e.g., a first end 110 and a second end 115) coupled with one another. The body 105 can be made from various metallic or non-metallic materials including, but not limited to, chromium, nickel, stainless steels, titanium, plastics, polyethylene, such as ultra-high-molecular-weight polyethylene, or ceramics.

The first end 110 can couple with a first joint component, such as a femoral component 112 (e.g., a component capable of coupling with a portion of the knee). The femoral component 112 can include two extensions that contact the first end 110 or another portion of the body 105, such as a spacer 113. The first end 110 can include one or more surfaces to receive the extensions of the femoral component 112. For example, the first end 110 can include at least one flat, curved, cratered, or annular surface to receive a portion of the first joint component, as described herein. The body 105 can include the spacer 113 between the first end 110 and the femoral component 112. For example, the spacer 113 can couple with the first end 110 and can include one or more surfaces to receive the extensions of the femoral component 112. The spacer 113 can include at least one flat, curved, cratered, or annular surface to receive a portion of the femoral component 112. The first end 110 can couple with various other joint components including, but not limited to, a humeral component or stem, a glenoid component, or pelvic component. The first end 110 can be made of one or more metallic materials. The spacer 113 can be made from one or more non-metallic materials, such as polyethylene.

The second end 115 can couple with a second joint component, such as a tibia component (e.g., a component capable of coupling with a portion of a tibia). The second end 115 can include or can couple with an attachment stem 117 to couple with a portion of the second joint component. For example, the attachment stem 117 can include a projection to couple with a portion of the tibia. The second end 115 can couple to various other joint components including, but not limited to, a glenoid sphere or component, humeral component, or a femoral component. The second end 115 can be made from various metallic materials.

The second end 115 can oppose the first end 110. For example, the body 105 can extend longitudinally between the first end 110 and the second end 115 in an axial direction (e.g., the thickness of the body 105 extends in a direction that is substantially parallel to the axial direction of the stem 117). In some examples, the first end 110 can couple with the second joint component and the second end 115 can couple with the first joint component (e.g., the first end 110 and the second end 115 may be interchangeable depending on the application).

The first end 110 can movably couple with the second end 115, as described herein. The position and orientation of the first end 110 can be defined or described by a first coordinate system 120. For example, the first coordinate system 120 can be defined by Cartesian coordinates (x,y,z) including an origin defined at some reference point on a surface of the first end 110, as shown in at least FIG. 1. Two axes (axes x and z), can be aligned with the first end 110. The first coordinate system 120 can be fixed with the first end 110 such that the first coordinate system 120 shifts and rotates as the first end 110 shifts and rotates in 3D space.

The position and orientation of the second end 115 can be defined by a second coordinate system 125. For example, the second coordinate system 125 can be defined by Cartesian coordinates (x′,y′,z′) including an origin defined at some reference point on a surface of the second end 115, as shown in at least FIG. 1. Two axes (axes x′ and z′) can be aligned with the second end 115. The second coordinate system 125 can be fixed with the second end 115 such that the second coordinate system 125 shifts and rotates as the second end 115 shifts and rotates in 3D space.

The first coordinate system 120 and the second coordinate system 125 can be related by a transformation T. The transformation T can define the translations and rotations between the two coordinate systems 120 and 125. Transformation T can, for example, be defined by three translation values and three angular rotation or quaternion values, by a 4×4 transformation matrix, or any other method of defining rigid transformations between two coordinate systems. The transformation T can be adjusted to conform to the patient's anatomy, as described herein. For example, the first end 110 can move axially or rotate relative to the second end 115.

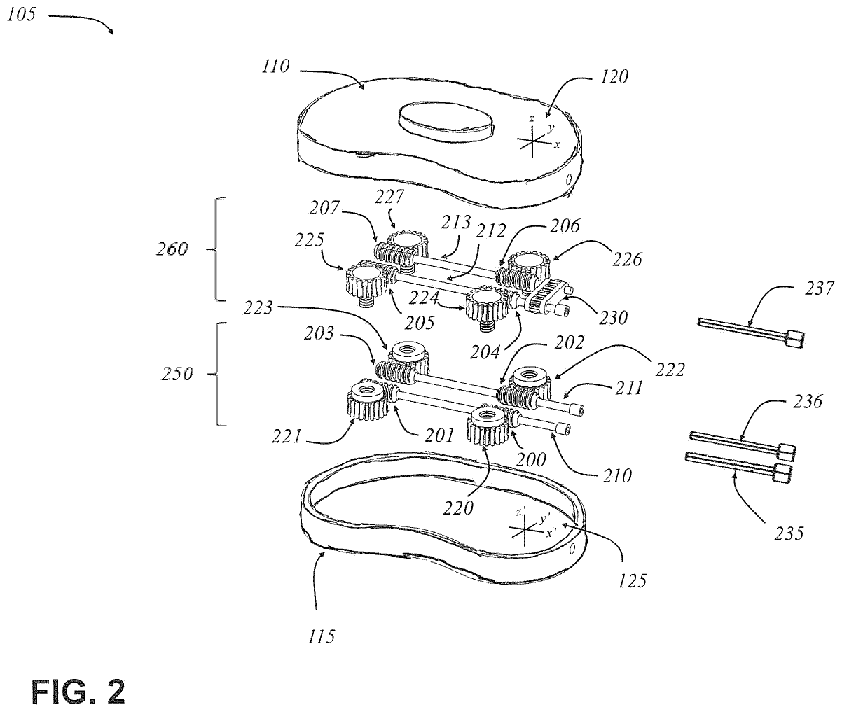

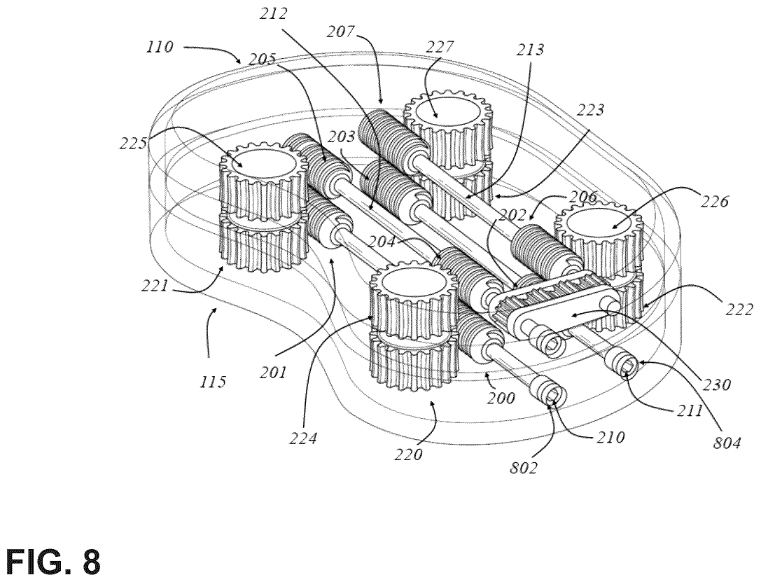

FIG. 2 illustrates a perspective exploded view of a portion of the body 105 of the joint implant device 100. FIG. 8 illustrates a perspective view of a portion of the body 105 of the joint implant device 100 assembled and with a transparent top and bottom surface. The body 105 can include at least two gear mechanisms. For example, the body 105 can include a first gear mechanism 250 and a second gear mechanism 260. The first gear mechanism 250 can include, for example, a plurality of gears and components positioned generally within a cavity defined by the second end 115 (e.g., gears 200, 201, 202, 203, 220, 221, 222, 223, and axles 210 and 211 described herein). The second gear mechanism 260 can include, for example, a plurality of gears and components positioned generally within a cavity defined by the first end 110 (e.g., gears 204, 205, 206, 207, 224, 225, 226, 227 and axles 212 and 213 described herein). As described herein, the first gear mechanism 250 can facilitate changing an axial distance between the first end 110 and the second end 115 (e.g., thereby increasing a thickness of the body 105) and can facilitate changing an angle between the first end 110 and the second end 115 about an axis (e.g., the x-axis or the x′-axis as shown in FIG. 1). The second gear mechanism 260 can facilitate changing an angle between the first end 110 and the second end 115 about a different axis (e.g., the y-axis or the y′-axis as shown in FIG. 1). The gear mechanisms can allow the body 105 to have at least one translational degree of freedom and at least two rotational degrees of freedom.

Referring to FIGS. 2 and 8, the body 105 (e.g., between the first and second gear mechanisms) can include at least two first gears. For example, the first gears can be or can include worm gears. The body 105 can include eight first gears 200, 201, 202, 203, 204, 205, 206 and 207. The first gears 200 and 201 can couple with a common axle 210, gears 202 and 203 can couple with a common axle 211, gears 204 and 205 can couple with a common axle 212, and gears 206 and 207 can couple with a common axle 213. Rotating the axles 210, 211, 212 and 213 can rotate the first gears they are coupled with simultaneously. The common axles can be or can include screws, rods, shafts, or other components coupleable to the gears. The first gears can be another gear, such as a pinion, bevel gear, screw gear, helical gear, or another gear.

The body 105 can include at least two second gears. For example, the second gears can be or can include worm wheels that engage with respective first gears. The body 105 can include eight second gears 220, 221, 222, 223, 224, 225, 226 and 227. The second gears 220, 221, 222, 223, 224, 225, 226 and 227 can engage with the first gears 200, 201, 202, 203, 204, 205, 206 and 207, respectively. For example, first gear 200 can engage with second gear 220, first gear 201 can engage with second gear 221, first gear 203 can engage with second gear 223, first gear 204 can engage with second gear 224, first gear 205 can engage with second gear 225, first gear 206 can engage with second gear 226 and first gear 207 can engage with second gear 227. The second gears 220, 221, 222, 223, 224, 225, 226 and 227 can be or can include various other types of gears including, but not limited to, a bevel gear, rack, helical gear, screw gear or another type of gear. The first gears 200, 201, 202, 203, 204, 205, 206, 207 can rotate in the same direction as each corresponding axle is rotated.

At least two second gears can include opposing male and female connecting portions such that at least two second gears can couple together. For example, second gear 220 can couple with second gear 224, 221 can couple with second gear 225, 222 can couple with second gear 226, and 223 can couple with second gear 227. The coupled pairs can include opposing male and female connecting portions to increase or decrease the distance between them in an axial direction (e.g., compress or expand a distance between the gears). For example, a first set of each pair (e.g., the lower second gears of each pair, such as second gears 220, 221, 222 and 223), can include a plurality of corrugations (e.g., threads) positioned along an inner female coupling portion and the upper members of each pair, (e.g., second gears 224, 225, 226 and 227), can include a plurality of corrugations positioned along an outer male portion (e.g., protrusion). The inner plurality of corrugations of each second gear can rotatably engage with the outer plurality of corrugations of its coupled second gear. For example, each female portion of the lower second gears can receive each of the male portion of the upper second gears. The plurality of corrugations (e.g., threads) of the female portion can correspond to the plurality of corrugations of the male portion such that rotation of the lower second gear causes the upper second gear to move in an axial direction (e.g., an axial direction of the male portion).

Each first gear can rotatably couple with each corresponding second gear such that rotation of a first gear (e.g., rotation of the axle) causes rotation of the second gear which causes a relative axial motion between the second gear and its respective coupled second gear as the plurality of corrugations included in the second gear aligns with and creates force against the plurality of corrugations included in the first gear. For example, rotation of second gear 220 can cause the plurality of threaded corrugations of the female portion to push or pull against the plurality of threaded corrugations of the male portion of the second gear 224 to cause the second gear 224 to move up or down relative to the second gear 220.

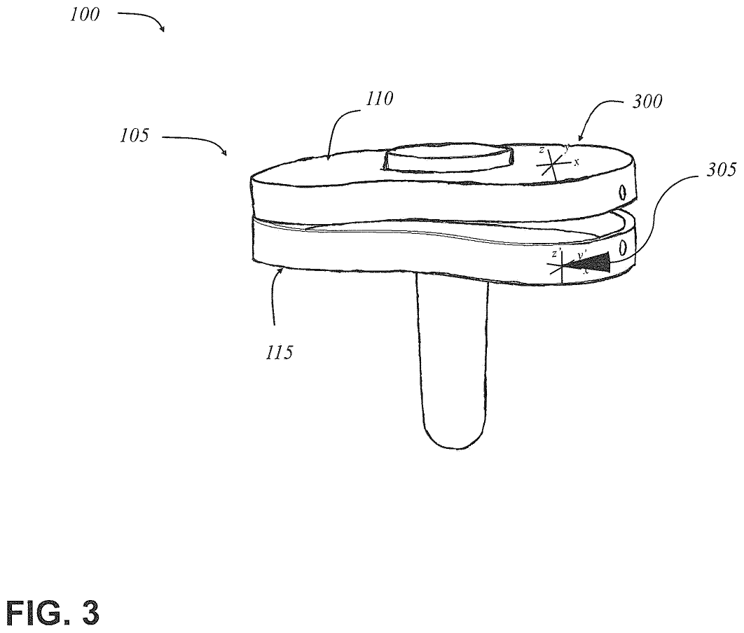

Second gears 220, 221, 222 and 223 can couple with the second end 115 and second gears 224, 225, 226 and 227 can couple with the first end 110 such that relative motion between any of the coupled gears can modify the translations and rotations between the first end 110 and the second end 115. This results in a different relationship between coordinate system 120 and coordinate system 125. This different relationship can be described using the transformation T. For example, FIG. 3 illustrates an example in which the gears have been adjusted such that a surface of the first end 110 and a surface of the second end 115 are no longer parallel. In this case, the coordinate system 120 is modified to a new coordinate system 300, and the transformation T is modified from an initial value T1 to a new value T2.

The transformation can occur by rotation of axles 210, 211, 212 and 213. Rotation of axle 210, for example, can simultaneously rotate first gear 200 and first gear 201. The rotation of first gear 200 can cause the second gear 220 to rotate. Rotation of the second gear 220 can cause an increase or decrease in the distance between the second gear 220 and the second gear 224 (e.g., by the threaded protrusion of the second gear 224 pushing or pulling on the threaded aperture of the second gear 220). Simultaneously, rotation of the first gear 201 can cause the second gear 221 to rotate. Rotation of the second gear 221 can cause an increase or decrease in the distance between the second gear 221 and the second gear 225 by the same amount.

Similarly, rotation of axle 211 may simultaneously rotate first gear 202 and first gear 203. The rotation of the first gear 203 can cause the second gear 223 to rotate. Rotation of the second gear 223 can cause an increase or decrease in the distance between the second gear 223 and the second gear 227. Simultaneously, rotation of the first gear 204 can cause the second gear 224 to rotate. Rotation of the second gear 224 can cause an increase or decrease in the distance between the second gear 224 and the second gear 226 by the same amount.

Referring to FIGS. 2 and 8, the axle 210 can couple with the lower portion of the body 105 (e.g., the second end 115) through a first through hole 802 and the axle 211 can couple with the lower portion of the body 105 (e.g., the second end 115) through a second through hole 804 such that the second end 115 is fixed to the axles 210, 211 and allows rotation of the axles 210, 211. The second gears 220, 221, 222 and 223 can couple with the second end 115 and the second gears 224, 225, 226 and 227 can couple with the first end 110 such that relative motion between any of the coupled gears causes the first end 110 or the second end 115 to move relative to one another. For example, each second gear can include at least one surface that engages (e.g., contacts, abuts, pushes against, attaches to) the first end 110 or the second end 115.

With this configuration, synchronous rotation of axle 210 and axle 211 can cause a change in axial distance (e.g., separation) between the first end 110 and second end 115 to increase or decrease the thickness of the body 105. For example, rotation of the axles 210, 211 at approximately the same time, direction, and rotational speed can cause a change in axial distance. Asynchronous rotation of axle 210 and axle 211 (e.g., either only rotation of one of the axles or rotation of both axles in at least one of a different direction, speed, or time) can cause a change in orientation (e.g., a rotation) between the first end 110 and the second end 115. For example, rotation of axle 210 can cause a first side portion of the first end 110 to increase (e.g., a side closest to the second gears 220, 221, 225, and 224) at a different rate than rotation of a second side portion of the first end 110 (e.g., a side closest to the second gears 222, 223, 226, and 227). This rotation can be about a single axis, such as the x-axis of the second or first coordinate system.

In order to rotate in the opposite direction (e.g., rotation about the y-axis), axle 212 and axle 213 can be used. For example, the first gear 204 and the second gear 224 can be oriented in an opposite rotation direction with respect to the first gear 205 and the second gear 225. For example, the first gear 204 and second gear 224 can include a first set of threads (e.g., right handed threads), while first gear 205 and second gear 225 can have an opposing second set of threads (e.g., left handed threads). Thus, if turned clockwise for example, rotation of axle 212 can increase the distance between second gears 220 and 224 while simultaneously decreasing the distance between second gears 221 and 225. If turned counterclockwise, rotation of axle 212 can decrease the distance between second gears 220 and 224 while simultaneously increasing the distance between second gears 221 and 225. Any of the first gears or second gears can have right handed or left handed threads such that the gears can rotate clockwise or counterclockwise.

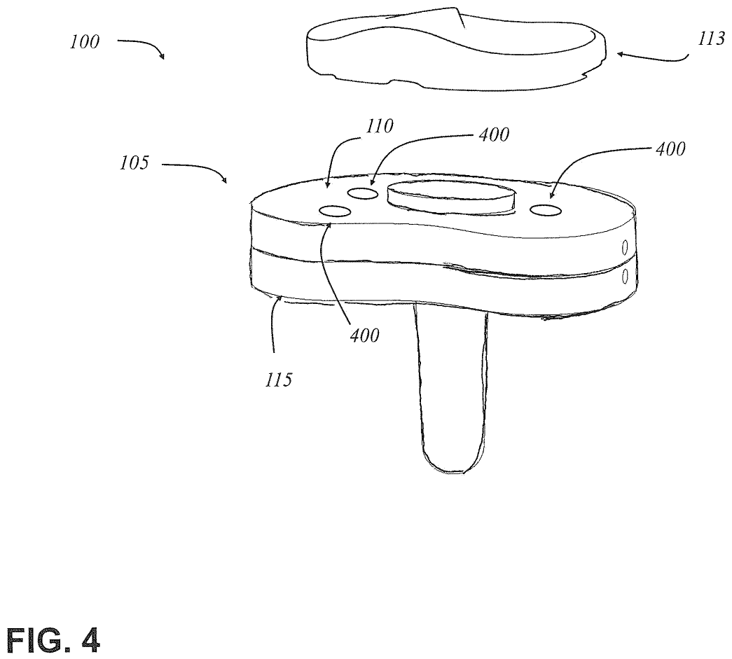

The axle 213 can be constrained to rotate in tandem with axle 212 (e.g., such that the axles 213 and 212 rotate simultaneously). For example the axle 212 and the axle 213 can couple with one another by a gear train 230, for example, or through a coordinated automatic control mechanism. The first gear 206 can be oriented in the same direction as the first gear 204, and the first gear 207 can be oriented in the same direction as first gear 205. Thus, the second gear 226 can follow the relative axial motion of the second gear 224, and the second gear 227 can follow the axial relative motion of second gear 225, such that rotation between the first end 110 and the second end 115 can occur upon rotation of the axle 212. This rotation can be about a single axis, for example the y-axis. FIG. 3 depicts an example of the body 105 after rotation of the axle 212 in a clockwise direction such that the first end 110 has been rotated relative to the second end 115 about the y-axis. FIG. 4 depicts an example of the body 105 after rotation of the axle 212 in a counterclockwise direction from the position show in FIG. 3. The first end 110 and the second end 115 can be sealed in a liquid-tight manner during application of the joint implant device 100.

Through various rotational combinations between axle 210, axle 211 and axle 212, the transformation T of the body 105 between the first end 110 and the second end 115 can be adjusted to correspond to changes in thickness and orientation between the first end and the second end of body 105. For example, the first end 110 and the second end 115 can rotate about the x-axis or y-axis at an angle between 0 degrees (e.g., 0.1 degrees) and 10 degrees (e.g., as shown by the angle between coordinate system 300 and coordinate system 305). This example is for illustrate purposes only. The first end 110 and the second end 115 can rotate substantially greater than 10 degrees. For example, the first end 110 and the second end 115 can rotate between 0 degrees and 90 degrees relative to one another about either the y-axis or the x-axis.

The thickness of the body 105 (e.g., the distance between the first end 110 and the second end 115 along the z-axis) can change, for example, between 0-20 mm (e.g., 0.1 and 20 mm). This example is for illustrate purposes only. The distance between the first end 110 and the second end 115 can change substantially greater than up to 20 mm. For example, the distance between the first end 110 and the second end 115 can change up to 500 mm or greater than 500 mm.

Rotations about one axis (e.g., the x-axis) can correspond to the pitch of the body 105, and rotation about another axis (e.g., the y-axis) can correspond to the roll of the body 105. In knee implants, rotations are often referred to as tilt, corresponding to rotation about the x-axis, and version, corresponding to rotation about the y-axis. Axles 210 and 211 can adjust the tilt, and axle 212 can adjust the version. Simultaneous movement of the axles 210 and 211 can adjust the thickness.

The joint implant device 100 can include at least one actuator operably coupled with a portion of each of the axles. For example, the actuator 235 can couple with axle 210, actuator 236 can couple with axle 211, and actuator 237 can couple with axle 212. For example, the actuators 235, 236, and 237 can include a fastener to engage with a portion of each axle. In some examples, the actuators 235, 236 and 237 can receive a portion of a hand tool (e.g., screw driver, wrench) to facilitate actuating (e.g., rotating) the axles 210, 211, and 212 relative to the body 105. For example, the axles 210, 211, and 212 can slidably receive a portion of the actuators 235, 236, and 237 such that rotation of the actuators 235, 236, and 237 causes rotation of the paired axle 210, 211, or 212 relative to the body 105. The actuators 235, 236, and 237 can include a rod, axle, or other component to facilitate rotatably coupling the axles 210, 211, and 212 with the first end 110 and the second end 115. The actuators 235, 236, and 237 can automatically actuate via one or more components of a data processing system or device application, as be described in greater detail herein. For example, one or more signals from a control system (e.g., data processing system described below) can cause the actuators 235, 236, and 237 to rotate a specific amount. The actuators 235, 236, and 237 can be positioned along portion of the joint implant device 100 such that the actuators 235, 236, 237 extend from an external side of the joint (e.g., an external side of a knee joint or other joints described herein) such that the actuators can be easily accessed.

The first gears and the second gears can automatically lock in place when not being actuated by the actuators 235, 236, 237. For example, friction between the teeth of the first gears and the teeth of the corresponding second gears can prevent the gears from rotating when the axles are not being rotated by an external force (e.g., by the actuators 235, 236, 237). Thus, the first end 110 and the second end 115 can automatically lock in position upon being actuated by the actuators 235, 236, 237 (e.g., the first end 110 and the second end 115 will not slide out of position).

The joint implant device 100 can detect various metrics, parameters, or characteristics. For example, the joint implant device 100 can include at least one component to detect a physical metric of the joint implant device 100 occurring proximate the first end 110 of the body 105, such as a load force (e.g., amplitude and direction of one or more points of contact between the first end 110 and the extensions of the femoral component 112), pressure, stability, or other similar physical metrics. For example, the joint implant device can include at least one sensor 400 positioned on or near the implant 100, such as on or near the first end 110. For example, as depicted in FIG. 4, three sensors 400 can detect translations or rotations. The sensors 400 can also be positioned on or near the spacer 113 or the second end 115. The joint implant device 100 can include any number of sensors 400. For example, the joint implant device 100 can include one sensor, two sensors, three sensors, or more than three sensors.

The sensors 400 can detect load forces placed on the first end 110 of the body 105. For example, the sensors 400 can detect load forces from the first joint component placed upon the first end 110 of the body 105. The sensors 400 can be or can include various types of sensors including, but not limited to, resistive sensors. For example, the sensors 400 can each detect or measure a temperature, pressure, displacement, force, vibration, or another characteristic occurring on or about the first end 110. In some examples, the sensors 400 can detect physical metrics of the joint implant device 100 based on a change in voltages or resistances of the sensors 400. For example, the sensors 400 can be or can include accelerometers, gyroscopes, geomagnetic sensors, potentiometers, resistive position transducers, resistive pressure transducers, thermistor, strain gauge, or other type of sensor.

The sensors 400 can detect, map, or otherwise determine a vector direction and amplitude of one or more load forces on the joint implant device 100. For example, each sensor 400 can detect a force relative to an axis of the sensor 400 (e.g., normal to the sensor).

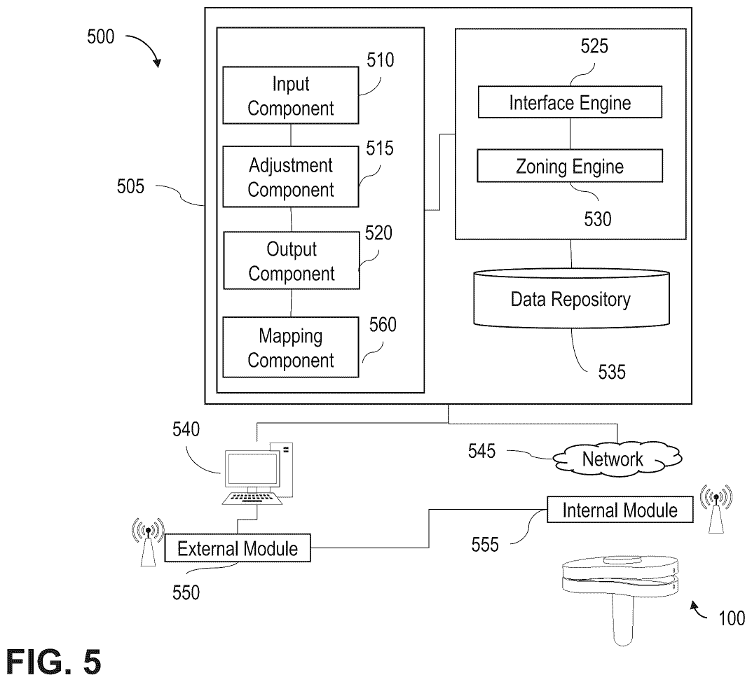

FIG. 5 depicts an example joint device system 500. For example, the joint device system 500 can facilitate evaluating the joint implant device 100. The joint device system 500 can include the joint implant device 100 and an internal module 555. For example, the internal module 555 can include the one or more sensors 400, and antenna, or a printed circuit board. The internal module 555 can receive at least one signal (e.g., data packet, an indication, etc.) from another portion of the joint device system 500, such as an external module 550. For example, the external module 550 can transmit signals to the internal module 555. The external module 550 can include one or more transceivers, phased array radar systems, or transmitters to transmit signals from the external module 550 to the internal module 555. In some examples, the external module 550 can include one or more frequency generators such that the external module 550 can transmit radio frequency signals (e.g., oscillations, electromagnetic waves, vibrations, etc.) in multiple directions. For example, the radio signals can reflect off of or be absorbed by one or more components of the joint implant device 100 to create a radio frequency feedback loop between the external module 550 and the internal module 555. In some examples, one or more signals, such as radio frequency signals, can cause the actuators 235, 236, 237 to rotate.

The internal module 555 can include various printed circuit boards (PCBs), wires, or other similar components that communicably couple with the one or more sensors 400. In some examples, the internal module 555 is passive (e.g., does not include a power source directly attached to the internal module). In some examples, the internal module and the sensors 400 can couple through various wires, cables, pins, or by direct contact. In some examples, the internal module 555 and the sensors 400 can couple through one or more wireless access points (e.g., via an antenna, Wi-Fi access point, etc.)

The joint implant device 100 can include an antenna. For example, the joint implant device 100 can include an antenna embedded within a portion of the body 105 of the joint implant device. The antenna can couple with a center portion of the body 105 such that the antenna is not exposed to an exterior of the body 105. The antenna can couple with an outer portion of the body 105, such as an outer surface of the first or second ends. For example, the antenna (e.g., a PCB antenna) can circumferentially surround one or more portions of the body 105 (e.g., spiral around, wrap around). In some examples, the antenna can integrally form with the body 105 during or after manufacturing of the fourth support. For example, the antenna may be casted inside a portion of the body 105.

The antenna can communicably couple to a portion of the one or more sensors 400. In some examples, the antenna can communicably couple with the internal module 555. In some examples, the antenna can communicably couple to the one or more sensors 400 to wirelessly couple the sensors 400 with the external module 550 separate from the joint implant device 100. For example, the external module 550 may include a patch, reinforcement, or the like to adhere to a portion of a patient's body (e.g., stick to the skin).

One or more components of the internal module 555 can receive the one or more signals from the external module 550. For example, the antenna integrally formed with the joint implant device 100 can receive the signals. In some examples, the external module 550 can transmit signals to the antenna such that the antenna and the external module 550 cause the internal module 555 (e.g., the sensors 400 and the PCB) to activate (e.g., turn on or off).

The sensors 400 can detect a load on the joint implant device 100. For example, the electrical properties of the sensors 400 can change or fluctuate when a load is placed on the joint implant device 100. The internal module 555 can detect a change or fluctuation of the sensors 400 and can adjust or change one or more properties of a radio frequency loop between the external module 550 and the internal module 555, which can create a change in a reflected signal from the internal module 555 to the external module 550.

The external module 550 can receive signals (e.g., data packet, an indication, etc.) from the internal module 555. For example, the external module 550 can receive reflected radio frequency signals from the internal module 555 in response to the transmitted signal from the external module 550. The external module 550 can be separate from the joint implant device 100. For example, the external module 550 can be physically positioned apart from the joint implant device 100.

The external module 550 can analyze the signals from the internal module 555 and determine, based on the analyzed signals, data corresponding to the load force on the joint implant device. In some examples, the external module 550 can communicably couple to one or more computing devices to transmit the data corresponding to the load force, the position of the body 105 (e.g., within a joint), or orientation in space of the joint implant device 100.

The joint device system 500 can include at least one data processing system 505. The data processing system 505 can include a variety of different components including, but not limited to, an input component 510, an adjustment component 515, an output component 520, and a mapping component 560. The data processing system 505 can include several engines including, but not limited to, an interface engine 525 and a zoning engine 530. The data processing system 505 can include at least one database, such as a data repository 535. While these components and engines are shown in FIG. 5, the data processing system 505 may include any number of device evaluating components or engines, including additional components or engines which may be incorporated into, supplement, or replace one or more of the engines shown in FIG. 5.

The data processing system 505 can include several components or engines. For example, the data processing system 505 can include components or engines to transmit or receive data from one or more remote sources (such as the computing devices, the external module 550, or the internal module 555). In some examples, communications device(s) may access the network 545 to exchange data with various other communications device(s) via cellular access, a modem, broadband, Wi-Fi, satellite access, etc. via the data processing system 505. The data processing system 505 may be any device(s), component(s), circuit(s), or other combination of hardware components designed or implemented to receive inputs or other signals for evaluating the joint implant device 100. For example, the data processing system 505 can receive inputs or other signals for evaluating the joint implant device 100 when the device 100 is being used within a joint, such as a shoulder joint.

The data processing system 505 can communicably couple with at least one client computing device 540. The data processing system 505 may communicably couple with the client computing device 540 via a communications link or network 545 (which may be or include various network connections configured to communicate, transmit, receive, or otherwise exchange data between addresses corresponding to the client computing device 540 and data processing system 505). The network 545 may be a Local Area Network (LAN), a Wide Area Network (WAN), a Wireless Local Area Network (WLAN), an Internet Area Network (IAN) or cloud-based network, etc. The network 545 may facilitate communication between the respective components of the joint device system 500.

The input component 510 of the data processing system 505 can obtain data based on the signals from the internal module 555 to the external module 550. For example, the input component 510 can obtain data transmitted from the external module 550 corresponding to one or more load forces and points of contact on the joint implant device 100. In some examples, the data transmitted from the external module 550 may include one or more voltage measurements detected by each sensor 400. For example, the input component 510 can calibrate each sensor (e.g., based on one or more calibration curves using one or more known loads) and can determine, based on the calibration, a corresponding force value for each detected voltage of each sensor 400 individually or simultaneously.

The adjustment component 515 can determine an indication to adjust (e.g., rotate, expand, or compress the body 105 by rotation of the axles 210, 211, or 212) the joint implant device 100. For example, the adjustment component 515 can determine an indication to adjust the length of the body 105 of the joint implant device 100, which may indicate to synchronously adjust both axles 210 and 211. In some examples, one or more tools can detect or localize the positions of interfaces of the axles 210, 211, and 212 such as an interface of the actuators, to adjust the first gears. For example, an arthroscopy can detect a location of an interface of the actuators 235, 236, 237 such that a user can locate and adjust the axles 210, 211 or 212 with their paired actuator 235, 236 or 327 (e.g., using a hand tool via a minimally invasive procedure). In some examples, the adjustment component 515 can transmit one or more signals to the joint implant device 100 (e.g., via the external module 550 or internal module 555) to cause rotation of one or more of the axles 210, 211 and 212. For example, the adjustment component 515 can cause the joint implant device 100 to automatically adjust based on an indication to adjust the device 100. In some examples, the adjustment component 515 can cause the joint implant device 100 to automatically adjust such that no surgical procedure is necessary to adjust the joint implant device 100 (e.g., via a non-invasive procedure). In some examples, the input component 510 can receive an input (e.g., a user input) from a computing device, such as a computer or mobile device, to adjust the joint implant device 100. For example, the adjustment component 515 can cause the joint implant device 100 to automatically adjust based on a user input on a mobile device application. In some examples, the data processing system 505 can include or can communicably couple with one or more AI systems to facilitate automatically adjusting the joint implant device 100.

For example, the adjustment component 515 can evaluate the data obtained by the input component 510 and can determine if the data corresponds to stable tension forces on the joint implant device 100. In some examples, the adjustment component 515 may determine, based on the signal received by the external module 550, that the joint implant device 100 includes too large of a force (e.g., has too much tension). For example, if a desired tension force of the joint implant device 100 (e.g., between the first joint component and the first end 110) is about 10 N and a detected force measurement greater than 500 N, the adjustment component 515 may determine that an adjustment of the joint implant device 100 is required. This adjustment can correspond to a change in thickness of the body 105 of the joint implant device 100 or a change in orientation (e.g., about the x-axis or y-axis described herein) of the body 105. This example is for illustrative purposes only and is not limiting to the scope of the joint implant device 100. The desired load force or the force at which the adjustment component 515 determines an indication to adjust the joint implant device 100 may occur at various other force measurements.

In some examples, the adjustment component 515 can determine an indication to maintain the joint implant device 100 in a position. For example, the external module 550 may receive signals from the internal module 555 that indicates one or more stable forces upon the joint implant device 100. By way of non-limiting example, if a desired tension force of the joint implant device 100 is about 10 N and a detected force measurement is about 10 N, the adjustment component 515 may determine that no adjustment of the joint implant device 100 is required. This example is for illustrative purposes only and is not limiting to the scope of the joint implant device 100. The desired load force or the force at which the adjustment component 515 determines an indication not to adjust the joint implant device 100 may occur at various other force measurements.

The adjustment component 515 can determine an indication to adjust or maintain the joint implant device 100 based on a location of the load forces (e.g., points of contact) of the joint implant device 100. For example, the mapping component 560 can map a location of the load forces of the joint implant device 100 on a portion of the first end 110 or spacer 113 of the joint implant device 100. The adjustment component 515 can determine, based on the detected location of a sum of forces detected by each sensor 400, whether to adjust or maintain the joint implant device 100. For example, if a detected location of a point of contact or force on the first end 110 or spacer 113 is far away from a center point of the extensions of the femoral component 112, the adjustment component 515 may determine an indication to adjust the joint implant device 100. If a detected location of the force on the first end 110 or spacer 113 is near the center points of the extensions of the femoral component 112, the adjustment component 515 may determine an indication to maintain the position of the joint implant device (e.g., not extend/retract the length of the body 105, not rotate/tilt the first end 110 relative to the second end 115).

In some examples, the adjustment component 515 may determine one or more tension forces (e.g., a soft-tissue tension force) that need to be applied to set the joint implant device 100 (e.g., specifically for each patient). For example, the adjustment component 515 can detect load forces (amplitude and direction) and determine an increase or decrease of the load forces (e.g., by adjusting the length of the body 105 or the orientation of the first end 110 relative to the second end 115) required to reach a desired load force. In some examples, the adjustment component 515 can determine tension forces applied to the joint implant device 100 to optimize a lifetime of the joint implant device 100. For example, the adjustment component 515 can determine tension or compression forces applied to the joint implant device 100 to limit fatigue, stress, or other factors to optimize use of the joint implant device 100 as long as possible (e.g., reduce fracture, breakage, or other damage of the device 100). The data processing system 505 can include a configuration similar to the computing system 600 described herein.

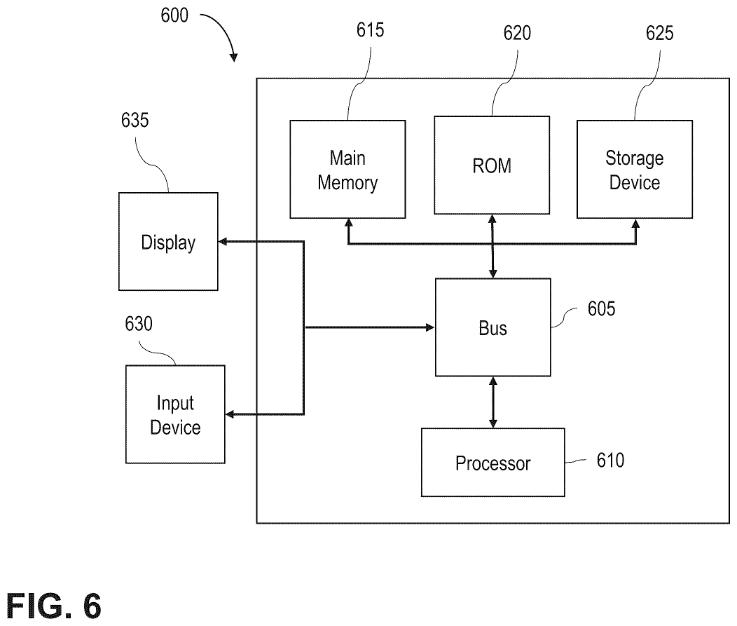

FIG. 6 illustrates a block diagram of an example computing system 600. The computer system or computing device 600 can include or be used to implement the system 500, or its components such as the data processing system 505. The computing system 600 includes a bus 605 or other communication component for communicating information and a processor 610 or processing circuit coupled to the bus 605 for processing information. The computing system 600 can also include one or more processors 610 or processing circuits coupled to the bus for processing information. The computing system 600 also includes main memory 615, such as a random access memory (RAM) or other dynamic storage device, coupled to the bus 605 for storing information, and instructions to be executed by the processor 610. The main memory 615 can be or include a data repository. The main memory 615 can also be used for storing position information, temporary variables, or other intermediate information during execution of instructions by the processor 610. The computing system 600 may further include a read only memory (ROM) 620 or other static storage device coupled to the bus 605 for storing static information and instructions for the processor 610. A storage device 625, such as a solid state device, magnetic disk or optical disk, can be coupled to the bus 605 to persistently store information and instructions. The storage device 625 can include or be part of the data repository.

The computing system 600 may be coupled via the bus 605 to a display 635, such as a liquid crystal display, or active matrix display, for displaying information to a user. An input device 630, such as a keyboard including alphanumeric and other keys, may be coupled to the bus 605 for communicating information and command selections to the processor 610. The input device 630 can include a touch screen display 635. The input device 630 can also include a cursor control, such as a mouse, a trackball, or cursor direction keys, for communicating direction information and command selections to the processor 610 and for controlling cursor movement on the display 635. The display 635 can be part of the data processing system 505, the client computing device 540 or other component of FIG. 5, for example.

The processes, systems and methods described herein can be implemented by the computing system 600 in response to the processor 610 executing an arrangement of instructions contained in main memory 615. Such instructions can be read into main memory 615 from another computer-readable medium, such as the storage device 625. Execution of the arrangement of instructions contained in main memory 615 causes the computing system 600 to perform the illustrative processes described herein. One or more processors in a multi-processing arrangement may also be employed to execute the instructions contained in main memory 615. Hard-wired circuitry can be used in place of or in combination with software instructions together with the systems and methods described herein. Systems and methods described herein are not limited to any specific combination of hardware circuitry and software.

Although an example computing system has been described in FIG. 6, the subject matter including the operations described in this specification can be implemented in other types of digital electronic circuitry, or in computer software, firmware, or hardware, including the structures disclosed in this specification and their structural equivalents, or in combinations of one or more of them.

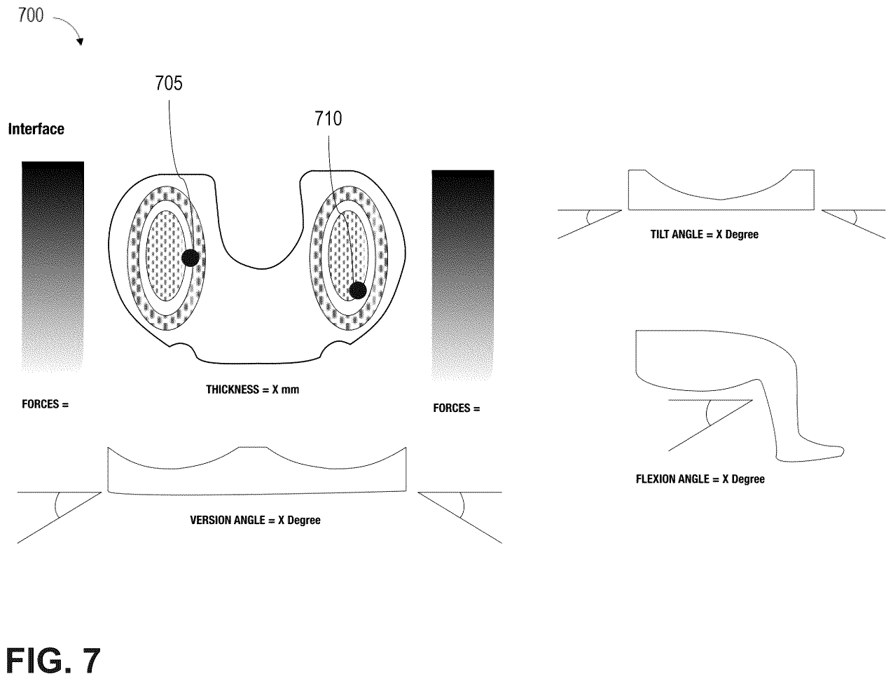

FIG. 7 depicts an example user interface 700 of the device system 500. For example, the user interface 700 can be displayed by the client computing device 540 or another device (e.g., computer, mobile device, or another device). As shown in FIG. 7, the user interface 700 can include a graphical representation of one or more points of contact 705, 710 between the projections of the femoral component 112 and the first end 110 or the spacer 113. FIG. 7 depicts an example in which the first point of contact 705 and the second point of contact 710 are slightly apart from a center point of the extensions of the femoral component 112 (e.g., away from the center point of the oval graphical representations). The user interface 700 can provide one or more numerical or other graphical recommendations. For example, the user interface 700 can include a thickness measurement (e.g., a distance between the first end 110 and the second end 115 along the z-axis). The user interface 700 can include a force measurement of one or more of the points of contact 705, 710 between the femoral component 112 and the first end 110 or spacer 113. The user interface 700 can include a measurement of the version angle (e.g., rotation about the y-axis), a measurement of the tilt angle (e.g., rotation about the x-axis), or a measurement of the flexion angle of the device 100 (e.g., rotation of the joint device relative to another portion of the body, such as flexion of the knee joint). The user interface 700 can include one or more notifications indicating an adjustment of the body 105. For example, the user interface 700 can include a graphical representation or a numerical indication indicating that the thickness of the body 105 should be increased or decreased or that an angle or orientation between the first end 110 and the second end 115 should be changed.

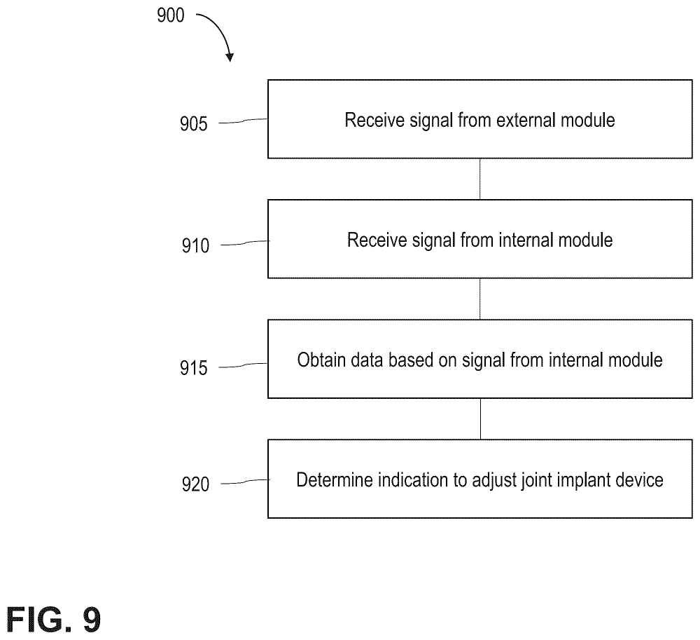

FIG. 9 illustrates an example of a method 900 of evaluating a joint implant device 100. The method 900 can include receiving signals from the external module 550, as depicted in act 905. For example, the internal module 555 of the joint implant device 100 can receive the signals. In some examples, the internal module 555 of the joint implant device 100 can receive the signals from the external module 550 to activate the joint implant device 100 (e.g., activate the sensors 400, power the internal module 555, etc.). As described herein, one or more portions of the joint implant device 100, such as the internal module 555, can reflect the signal away from the joint implant device 100, such as towards the external module 550.

The method 900 can include receiving signals from the internal module 555, as depicted in act 910. For example, the external module 550 can receive the signals from the internal module 555. In some examples, the external module 550 can receive reflected frequency signals from the internal module 555 in response to a first frequency signal from the external module 550 to the internal module 555. In some examples, the reflected signal corresponds to a detected load or movement of the first end 110 of the joint implant device 100.

The method 900 can include obtaining data based on the signals received from the internal module 555, as depicted in act 915. For example, the input component 510 of the data processing system 505 can obtain one or more data, or inputs, from the external module 550 based on the reflected signals from the internal module 555. In some examples, the data corresponds to a detected load on the first end 110 of the joint implant device 100. For example, the input component 510 may obtain one or more voltage or resistance measurements of the sensors 400 corresponding to a change in load on the first end 110 of the joint implant device 100.

The method 900 can include determining an indication to adjust the joint implant device 100, as depicted in act 920. For example, one or more components of the data processing system 505, such as the adjustment component 515, can determine whether an adjustment of the length of the body 105 or the orientation of the first end 110 or second end 115 of the joint implant device 100 is required. For example, if a tension force of the joint implant device 100 is too high (e.g., greater than 10% more than a desired force, for example), the adjustment component 515 can determine an indication to adjust the length (e.g., thickness) or orientation (e.g., angle of orientation between the first end 110 and the second end 115) of the body 105 of the joint implant device 100. If a tension force of the joint implant device 100 is too low (e.g., less than 10% less than a desired force, for example), the adjustment component 515 can determine an indication to adjust the length (e.g., thickness) or orientation (e.g., angle of orientation between the first end 110 and the second end 115) of the body 105 of the joint implant device 100. In some examples, the first gears can adjust based on the indication to adjust the length (e.g., thickness) or orientation (e.g., angle of orientation between the first end 110 and the second end 115) of the body 105. For example, a user can adjust the length or orientation of the body 105 of the joint implant device 100 by causing the one or more of the axles 210, 211, 212 to rotate (e.g., by using a hand tool). As described herein, the rotation of the axles 210, 211, 212 can cause the length of the body 105 to extend or retract or the orientation between the first end 110 and the second end 115 to be adjusted.

In some examples, the adjustment component 515 can determine to maintain the joint implant device 100 in a position. For example, if a detected tension force on the first end 110 of the joint implant device 100 is about equal to a desired tension force (e.g., within 10% of the desired force, for example), the adjustment component 515 can determine that an adjustment to the length or orientation of the body 105 is not required.

The computing system(s) described herein can include clients and servers. A client and server are generally remote from each other and typically interact through a communication network. The relationship of client and server arises by virtue of computer programs running on the respective computers and having a client-server relationship to each other. In some implementations, a server transmits data (e.g., data packets) to a client device (e.g., for purposes of displaying data to or receiving user input from a user interacting with the client device). Data generated at the client device (e.g., a result of the user interaction) can be received from the client device at the server.

The separation of various system components does not require separation in all implementations, and the described program components can be included in a single hardware or software product. For example, the components described herein can be a single component, app, or program, or a logic device having one or more processing circuits, or executed by one or more processors of the data processing system(s).

Some of the description herein emphasizes the structural independence of the aspects of the system components. Other groupings or components that execute similar overall operations are understood to be within the scope of the present application. Modules or components can be implemented in hardware or as computer instructions on a non-transient computer readable storage medium, and modules can be distributed across various hardware or computer based components.

The systems described above can provide multiple ones of any or each of those components and these components can be provided on either a standalone system or on multiple instantiation in a distributed system. In addition, the systems and methods described above can be provided as one or more computer-readable programs or executable instructions embodied on or in one or more articles of manufacture. The article of manufacture can be cloud storage, a hard disk, a CD-ROM, a flash memory card, a PROM, a RAM, a ROM, or a magnetic tape. In general, the computer-readable programs can be implemented in any programming language, such as LISP, PERL, C, C++, C#, PROLOG, or in any byte code language such as JAVA. The software programs or executable instructions can be stored on or in one or more articles of manufacture as object code.

Example and non-limiting module implementation elements include sensors providing any value determined herein, sensors providing any value that is a precursor to a value determined herein, datalink or network hardware including communication chips, oscillating crystals, communication links, cables, twisted pair wiring, coaxial wiring, shielded wiring, transmitters, receivers, or transceivers, logic circuits, hard-wired logic circuits, reconfigurable logic circuits in a particular non-transient state configured according to the module specification, any actuator including at least an electrical, hydraulic, or pneumatic actuator, a solenoid, an op-amp, analog control elements (springs, filters, integrators, adders, dividers, gain elements), or digital control elements.

The subject matter and the operations described herein can be implemented in digital electronic circuitry, or in computer software, firmware, or hardware, including the structures disclosed in this specification and their structural equivalents, or in combinations of one or more of them. The subject matter described herein can be implemented as one or more computer programs, e.g., one or more circuits of computer program instructions, encoded on one or more computer storage media for execution by, or to control the operation of, data processing apparatuses. The program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices include cloud storage). The operations described in this specification can be implemented as operations performed by a data processing apparatus on data stored on one or more computer-readable storage devices or received from other sources.

The terms “computing device”, “component” or “data processing apparatus” or the like encompass various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, app, script, or code) can be written in any form of programming language, including compiled or interpreted languages, declarative or procedural languages, and can be deployed in any form, including as a stand-alone program or as a module, component, subroutine, object, or other unit suitable for use in a computing environment. A computer program can correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit). Devices suitable for storing computer program instructions and data can include non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

The subject matter described herein can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or a combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or embodiment, and references to “an implementation,” “some implementations,” “one implementation” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

Systems and methods described herein may be embodied in other specific forms without departing from the characteristics thereof. For example, the joint implant device 100 can be implanted in a shoulder joint, a knee joint, an ankle joint, or a hip joint. Further relative parallel, perpendicular, vertical or other positioning or orientation descriptions include variations within +/−10% or +/−10 degrees of pure vertical, parallel or perpendicular positioning. References to “approximately,” “about” “substantially” or other terms of degree include variations of +/−10% from the given measurement, unit, or range unless explicitly indicated otherwise. Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

What is claimed is:

1. A joint implant device, comprising:
a body having a first end to couple with a first joint component and a second end to couple with a second joint component, the body comprising:
a first gear mechanism to cause a change in axial distance between the first end and the second end of the body; and
a second gear mechanism to cause a change in angle between the first end and the second end of the body about an axis.

2. The joint implant device of claim 1, comprising:
the axis includes a first axis;
the first gear mechanism to cause a change in angle between the first end and the second end of the body about a second axis; and
the second axis perpendicular to the first axis.

3. The joint implant device of claim 1, comprising:
a sensor coupled with the body; and
the sensor to detect a load force on the body.

4. The joint implant device of claim 1, comprising:
a spacer coupled with the first end of the body; and
the spacer to couple with the first joint component.

5. The joint implant device of claim 1, comprising:
the change in axial distance in the range of 0.1 to 20 mm.

6. The joint implant device of claim 1, comprising:
the change in angle in the range of 0.1 to 10 degrees.

7. The joint implant device of claim 1, comprising:
each of the first gear mechanism and the second gear mechanism to actuate via an actuator.

8. A joint implant device, comprising:
a prosthesis body having a first end to couple with a first joint component and a second end to couple with a second joint component, the prosthesis body comprising:
a first set of gears coupled with a first axle;
a second set of gears coupled with a second axle;
a third set of gears coupled with a third axle; and
a fourth set of gears coupled with a fourth axle;
wherein synchronous rotation of the first axle and the second axle causes a change in axial distance between the first end and the second end of the prosthesis body;
wherein asynchronous rotation of the first axle and the second axle causes a change in angle between the first end and the second end of the prosthesis body about a first axis; and
wherein rotation of the third axle and rotation of the fourth axle causes a change in angle between the first end and the second end of the prosthesis body about a second axis that is perpendicular to the first axis.

9. The joint implant device of claim 8, comprising:
the prosthesis body having at least two rotational degrees of freedom.

10. The joint implant device of claim 8, comprising:
the prosthesis body having at least one translational degree of freedom.

11. The joint implant device of claim 8, comprising:
the third axle coupled with the fourth axle such that the fourth axle rotates dependently with the third axle.

12. The joint implant device of claim 8, comprising:
the first axle to rotate independently from the second axle.

13. The joint implant device of claim 8, comprising:
a sensor coupled with the prosthesis body; and
the sensor to detect a load force on the prosthesis body.

14. The joint implant device of claim 8, comprising:
a spacer coupled with the first end of the prosthesis body; and
the spacer to couple with the first joint component.

15. The joint implant device of claim 8, comprising:
the change in axial distance in the range of 0.1 to 20 mm.

16. The joint implant device of claim 8, comprising:
the change in angle in the range of 0.1 to 10 degrees.

17. The joint implant device of claim 8, comprising:
each of the first axle and the second axle to actuate via an actuator.

18. A method, comprising:
receiving, via an internal module of a joint implant device, a first signal from an external module to activate the internal module;
detecting, via the internal module of the joint implant device, a physical metric of the joint implant device;
receiving, via the external module, a second signal from the internal module in response to the first signal;
obtaining, via an input component of a data processing system, data based on the second signal corresponding to the physical metric detected by the internal module of the joint implant device from the external module communicably coupled with the internal module of the joint implant device; and
determining, via an adjustment component of the data processing system, an indication to adjust at least one of a thickness or an orientation of the joint implant device based on the second signal.

19. The method of claim 18, comprising:
the physical metric comprising a load force on a portion of the joint implant device.

20. The method of claim 18, comprising:
the external module comprising at least one of a transceiver, a phased array radar system, or a transmitter; and
the internal module comprising at least one of a sensor, an antenna, or a printed circuit board.
